(19) United States
(12) Patent Application Publication
(10) Pub. No.: US 2026/0241005 A1
(43) Pub. Date: Aug. 20, 2026

(54) COMBINATION THERAPY FOR THE TREATMENT OF IMMUNE CHECKPOINT BLOCKADE RESISTANT CANCERS

(71) Applicants: Wisconsin Alumni Research Foundation, Madison, WI (US); City of Hope, Duarte, CA (US)

(72) Inventors: Deric L Wheeler, Blue Mounds, WI (US); Bridget Crossman, Verona, WI (US); Ravi Salgia, Pasadena, CA (US)

(73) Assignees: Wisconsin Alumni Research Foundation, Madison, WI (US); City of Hope, Duarte, CA (US)

(21) Appl. No.: 19/533,747

(22) Filed: Feb. 9, 2026

Related U.S. Application Data
(60) Provisional application No. 63/760,394, filed on Feb. 19, 2025.

(51) Int. Cl. A61K 45/06; A61K 31/496; A61K 31/517; A61K 31/519; A61K 39/395

(52) U.S. Cl. CPC A61K 45/06; A61K 31/496; A61K 31/517; A61K 31/519; A61K 39/3955

(57) ABSTRACT

Described herein are methods of treating a patient with a cancer that is resistant to immune checkpoint inhibitor therapy such as anti-PD-1 therapy, anti-PD-L1 therapy and/or anti-CTLA-4 therapy by administering to the patient a combination including an EPHA2 receptor tyrosine kinase inhibitor, and an immune checkpoint inhibitor, wherein the immune checkpoint inhibitor includes an anti-PD-1 inhibitor, an anti-PD-L1 inhibitor, an anti-CTLA-4 inhibitor, or a combination thereof. In an aspect, a method of treating HNSCC that is resistant to anti-PD-1/PD-L1 therapy includes administering a combination of ensartinib and pembrolizumab.

### CROSS-REFERENCE TO RELATED APPLICATIONS

This application claims priority to U.S. Provisional Application 63/760,394 filed on Feb. 19, 2025, which is incorporated herein by reference in its entirety.

### FIELD OF THE DISCLOSURE

The present disclosure is related to a combination therapy for the treatment of cancers, such as head and neck cancers, that overcomes resistance to immune checkpoint inhibitor therapy such as anti-PD-1/PD-L1 therapy.

### BACKGROUND

Squamous cell carcinomas of the head and neck (HNSCC), which arise in the oral cavity, oropharynx, larynx and hypopharynx, are a major public health concern. HNSCC is one of the top 10 most common cancers, with a 5-year survival rate of 50% or less. The incidence of HNSCC is rising with the increasing incidence of HPV-positive oropharyngeal cancer. There is an urgent need to develop new approaches to achieve durable cure and relapse-free survival.

Immune check point blockers (ICB), such as pembrolizumab and nivolumab (anti-PD-1), have demonstrated potent anti-tumor activity in a subset of HNSCC patients. These T cell targeted therapeutics can re-activate anti-tumor T cell responses; however, one-year survival and response rates of anti-PD-1 in HNSCC were only 36% and 13%, respectively, in a Phase 3 clinical trial (Cohen, E. E. W. et al. “Pembrolizumab versus methotrexate, docetaxel, or cetuximab for recurrent or metastatic head-and-neck squamous cell carcinoma (KEYNOTE-040): a randomised, open-label, phase 3 study”, Lancet 393, 156-167, doi:10.1016/S0140-6736(18)31999-8 (2019)). This highlights the urgent need to identify novel therapeutic options to increase the effectiveness of ICB for patients that do not respond to anti-PD-1/PD-L1 therapy. This disclosure satisfies this need and provides related advantages as well.

### BRIEF SUMMARY

In an aspect, a method of treating a patient with a cancer that is resistant to anti-PD-1 therapy, anti-PD-L1 therapy, anti-CTLA-4 therapy, or a combination thereof, comprises administering to the patient a combination comprising an EPHA2 receptor tyrosine kinase inhibitor and immune checkpoint inhibitor, wherein the immune checkpoint inhibitor comprises an anti-PD-1 inhibitor, an anti-PD-L1 inhibitor, an anti-CTLA-4 inhibitor, or a combination thereof.

In another aspect, a method of treating HNSCC that is resistant to anti-PD-1 therapy or anti-PD-L1 therapy comprises administering a combination comprising ensartinib and pembrolizumab.

### BRIEF DESCRIPTION OF THE DRAWINGS

FIGS. 1A-I show that ensartinib sensitizes tumors to anti-PD-L1 treatment. 1A) Whole cell lysate was harvested from MOC2 untreated or MOC2 ensartinib-treated cells and immunoprecipitated with anti-EPHA2 antibody. Phospho-tyrosine and EPHA2 levels were determined by immunoblot. 1B) Illustration of MOC2 experimental timeline and treatment strategy. 1C) MOC2 cells were injected onto the flanks of C57bl/6 mice. Tumor-bearing mice were treated with either vehicle, anti-PD-L1 (10 mg/kg; every 3-4 days) therapeutic antibody, ensartinib (25 mg/kg; BID), or the combination of anti-PD-L1 antibody and ensartinib. Mean values and SEMs are shown (n=14 tumors per group). 1D) Spaghetti plots demonstrating growth curves for each individual tumor. 1E) Tumor-infiltrating lymphocytes (TILs) were analyzed by flow cytometry. Fold change relative to vehicle control group and SEMs are shown (n=8 tumors per group). *P<0.05; **P<0.01; ns=not significant. 1F) Whole cell lysate was harvested from FMOC1 untreated or FMOC1 ensartinib-treated cells and immunoprecipitated with anti-EPHA2 antibody. Phospho-tyrosine and EPHA2 levels were determined by immunoblot. 1G) Illustration of FMOC1 experimental timeline and strategy. 1H) FMOC1 cells were injected onto the flanks of FVB/NJ mice. Tumor-bearing mice were treated with either vehicle, anti-PD-L1 (10 mg/kg; every 3-4 days) therapeutic antibody, ensartinib (25 mg/kg; BID), or the combination of anti-PD-L1 and ensartinib. Mean values and SEMs are shown (n=5-7 tumors per group). *P<0.05; ns=not significant. 1I) Spaghetti plots demonstrating growth curves for each individual tumor. Dashed lines indicate tumors that regressed completely without recurrence.

FIGS. 2A-D show EPHA2 blockade with ensartinib dampens DDR1 expression and prevents buildup of dense collagen structures. 2A) Whole cell lysate was collected from MOC2 untreated or MOC2 ensartinib-treated cells and immunoblotted for total and phosphorylated DDR1. 2B) Representative images of collagen structures in vehicle and ensartinib-treated MOC2 tumors as analyzed by PicroSirius Red, COL1A1 staining, and Second Harmonic Generation (SHG) imaging. 2C) Mean intensity of Picro Sirius red and COL1A1 signal was analyzed in FIJI. 2D) Number of collagen fibers was analyzed by running SHG images through the CT-FIRE analysis pipeline.

The above-described and other features will be appreciated and understood by those skilled in the art from the following detailed description, drawings, and appended claims.

### DETAILED DESCRIPTION

Resistance to immune checkpoint inhibitors such as anti-PD-1/PD-L1 therapy is a persistent problem in the management of patients with head and neck squamous cell carcinoma (HNSCC) and other cancers. Described herein is the role of the receptor tyrosine kinase EPHA2 in mediating this resistance. Targeting EPHA2 receptor tyrosine kinase with an EPHA2 inhibitor such as ensartinib in combination with anti-PD-1/PD-L1 therapy overcomes this resistance. In an aspect, the methods described herein provide a strategy to overcome pembrolizumab resistance which is observed in as many as 85% of HNSCC patients.

In an aspect, a method of treating a patient with a cancer that is resistant to anti-PD-1 therapy, anti-PD-L1 therapy, anti-CTLA-4 therapy, or a combination thereof, comprises administering a combination comprising an EPHA2 receptor tyrosine kinase inhibitor, and an immune checkpoint inhibitor, wherein the immune checkpoint inhibitor comprises an anti-PD-1 inhibitor, an anti-PD-L1 inhibitor, an anti-CTLA-4 inhibitor, or a combination thereof.

As used herein, combination therapy means that two or more medications are used simultaneously to treat the same condition. When the two or more medications are used simultaneously, the patient has the beneficial therapeutic effect of both agents, however, both agents do not need to be administered on the same schedule. For example, oral medications can be administered daily, while intravenous medications such as monoclonal antibodies may be administered once per week, 2 weeks, 3 weeks, 4 weeks, etc. Thus, simultaneous administration does not mean the 2 medications are administered at the same time, but rather the therapeutic effect of both medications are present in the patient.

In an aspect, the combination therapy described herein improves clinical endpoints in checkpoint inhibitor-resistant cancers. Exemplary clinical endpoints include overall survival (OS), overall response rate (ORR), and a reduction in tumor progression. OS is the length of time from the start of treatment to the patient's death. ORR is the percentage of patients who have a complete or partial response to treatment. In clinical trials, the reduction in tumor progression can be referred to as the progression-free survival (PFS) and measures the length of time a patient lives without their cancer progressing. In an aspect, the combination therapy described herein can provide a 40% to 50% improvement in tumor progression.

In an aspect, the EPHA2 receptor tyrosine kinase inhibitor comprises ensartinib, dasatinib, targefrin, regorafenib, ALW-II-41-27 ((N-[4-[(4-ethyl-1-piperazinyl)methyl]-3-(trifluoromethyl)phenyl]-4-methyl-3-[[[5-(2-thienyl)-3-pyridinyl]carbonyl]amino]-benzamide)), or a combination thereof, specifically ensartinib. In an aspect, the EPHA2 receptor tyrosine kinase inhibitor is orally administered. In another aspect the EPHA2 receptor tyrosine kinase inhibitor is orally administered daily.

Ensartinib (Ensacove™, Xcovery Holding Inc.) is an approved first line treatment for ALK-positive non-small-cell lung cancer (NSCLC). Ensartinib efficacy was evaluated in an open-label, randomized, multicenter trial in 290 patients with locally advanced or metastatic ALK-positive NSCLC who had not previously received an ALK-targeted therapy (eXALT3). Patients were randomized to receive ensartinib or crizotinib. Efficacy outcome measures were progression-free survival (PFS) and overall survival (OS). Ensartinib demonstrated a statistically significant PFS improvement compared to crizotinib.

Ensartinib is described in U.S. Pat. No. 9,126,947, incorporated herein by reference for its disclosure of inhibitors of ALK protein kinases. U.S. Pat. No. 10,899,744, incorporated by reference, describes crystalline forms of ensartinib. The recommended dose of ensartinib is 225 mg orally once daily with or without food.

Dasatinib (Sprycel®) is approved to treat Philadelphia chromosome-positive chronic myeloid leukemia in the chronic phase. Dasantinib is a multi-targeted tyrosine kinase inhibitor which has significant inhibitory activity on EPHA2. The recommended adult dose of dasatinib is 100 to 140 mg orally once daily with or without food.

Regorafenib (Stivarga®) is an inhibitor of multiple kinases currently approved for the treatment of colon or rectal cancer, gastrointestinal stromal tumor, and hepatocellular carcinoma. The recommended dose is 160 mg orally once daily for the first 21 days of a 28 day cycle.

Targefrin and ALW-II-41-27 are investigational EPHA2 inhibitors.

As used herein, a PD-1 inhibitor is a checkpoint inhibitor molecule that blocks the activity of the PD-1 immune checkpoint. A PD-L1 inhibitor is a checkpoint inhibitor molecule that blocks the activity of the PD-L1 immune checkpoint. PD-1 is a checkpoint protein expressed on the surface of immune cells, which can attach to PD-L1, a protein expressed on the surface of immune and cancer cells. When PD-1 binds to PD-L1, immune cells are prevented from killing cancer cells. Blocking the PD-1/PD-L1 interaction with an immune checkpoint inhibitor allows the T cells to kill tumor cells.

In an aspect, the anti-PD-1 or anti-PD-L1 inhibitor comprises an anti-PD-1 or anti-PD-L1 monoclonal antibody.

Exemplary PD-1 inhibitors include, but are not limited to, pembrolizumab (Keytruda®), nivolumab (Opdivo®), cemiplimab (Libtayo®), tislelizumab (Tevimbra®), dostarlimab (Jemperli®), retifanlimab (Zynyz®), and toripalimab (Loqtorzi®). Exemplary PD-L1 inhibitors include, but are not limited to, avelumab (Bavencio®), durvalumab (Imfinzi®), cosibelimab (Unloxcyt®), and atezolizumab (Tecentriq®).

Pembrolizumab is an intravenous formulation approved to treat melanoma, NSCLC, and HNSCC, among others. In the treatment of HNSCC, pembrolizumab can be combined with platinum and fluorouracil, or used as a single agent, for example, for patients with tumors expressing PD-L1, such as tumors having a combined positive score (CPS)≥1. Typical dosages are 200 mg every 3 weeks, or 400 mg every 6 weeks.

Nivolumab is an intravenous formulation approved to treat melanoma, NSCLC, and HNSCC, among others. In the treatment of HNSCC, nivolumab is typically administered with disease progression on or after platinum-based therapy. Typical dosages are 240 mg every 2 weeks, or 480 mg every 4 weeks.

Cemiplimab is an intravenous formulation approved to treat basal cell carcinoma and NSCLC, among others. Typical dosages are 350 mg administered every 3 weeks.

Tislelizumab is an intravenous formulation approved to treat esophageal cancer and gastric cancer. Typical dosages are 200 mg administered every 3 weeks.

Dostarlimab is an intravenous formulation approved to treat endometrial cancer and mismatch repair deficient recurrent or advanced solid tumors. Typical dosages are 500 mg administered every 3 weeks, followed by 1,000 mg administered every 6 weeks.

Retifanlimab is an intravenous formulation approved for the treatment of Merkel cell carcinoma. Typical dosages are 500 mg administered every 4 weeks.

Toripalimab is an intravenous formulation indicated for the treatment of nasopharyngeal carcinoma. Typical dosages are 240 mg administered every 3 weeks.

Avelumab is an intravenous formulation approved for the treatment of Merkel cell carcinoma, urothelial carcinoma, and renal cell carcinoma. Typical dosages are 800 mg administered every 2 weeks.

Durvalumab is an intravenous formulation approved for the treatment of NSCLC, small-cell lung cancer, hepatocellular carcinoma, and endometrial cancer. Typical dosages are 1,500 mg administered every 3 or 4 weeks.

Cosibelimab is an intravenous formulation approved for the treatment of cutaneous squamous cell carcinoma. Typical dosages are 800 mg administered every 2 weeks.

Atezolizumab is an intravenous formulation approved for the treatment of NSCLC, small cell lung cancer, hepatocellular carcinoma, melanoma, and alveolar soft part sarcoma. Typical dosages are 840 mg administered every 2 weeks, 1,200 mg administered every 3 weeks, or 168 mg administered every 4 weeks.

As used herein, a CTLA-4 inhibitor is a checkpoint inhibitor molecule that blocks the activity of the CTLA-4 immune checkpoint. Blockade of CTLA-4 increases the ability of T cells to kill cancer cells.

In an aspect, the CTLA-4 inhibitor comprises an anti-CTLA-4 monoclonal antibody.

Exemplary CTLA-4 inhibitors include ipilimumab, tremelimumab, and combinations thereof.

Ipilimumab (Yervoy®) is a monoclonal antibody that binds CTLA-4 and is available in an intravenous formulation approved for the treatment of melanoma, renal cell carcinoma, colorectal cancer, hepatocellular carcinoma, NSCLC, malignant pleural mesothelioma, and esophageal cancer. Typical dosages are 3 mg/kg every 3 weeks, after which time the frequency may be reduced.

Tremelimumab (Imjudo®) is a monoclonal antibody that binds CTLA-4 and is available in an intravenous formulation approved for the treatment of unresectable hepatocellular carcinoma and NSCLC. Typical dosages are 300 mg as a single dose, or 75 mg every 3 weeks.

In an aspect, the cancer is resistant to anti-PD-1 and/or PD-L1-therapy. Exemplary cancers resistant to anti-PD-1 and/or anti-PD-L1-therapy include head and neck cancers, melanoma, NSCLC, Hodgkins lymphoma, Merkel cell carcinoma, microsatellite instability-high (MSI-H) and mismatch repair deficiency (dMMR), colorectal cancer, gastric cancer, hepatocellular carcinoma, cervical cancer, small cell lung cancer, cutaneous squamous cell carcinoma, alveolar soft part carcinoma, basal cell carcinoma, biliary tract carcinoma, bladder cancer, breast cancer, mismatch repair deficiency (dMMR) solid cancers, endometrial cancer, esophageal cancer, malignant pleural mesothelioma (MPM), mesothelioma, nasopharyngeal cancer, non-Hodgkin's lymphoma, renal cell carcinoma, and cancers with a high tumor burden (TMB-high cancers), specifically HNSCC.

TABLE 3
FDA approved anti-PD-1/PD-L1 treatments

| Tumor Type | Drug |
|---|---|
| Melanoma | Pembrolizumab, Nivolumab, |
| | Atezolizumab |
| NSCLC | Pembrolizumab, Nivolumab, |
| | Atezolizumab, Durvalumab, |
| | Cemiplimab |
| Hodgkins Lymphoma | Pembrolizumab, Nivolumab |
| HNSCC | Pembrolizumab, Nivolumab |
| Merkel Cell Carcinoma | Avelumab, Pembrolizumab |
| MSI-H/dMMR | Pembrolizumab |
| Colorectal | Pembrolizumab, Nivolumab |
| Gastric | Pembrolizumab, Nivolumab |
| Hepatocellular | Pembrolizumab, Nivolumab, |
| | Atezolizumab, Durvalumab |
| Cervical | Pembrolizumab |
| SCLC | Pembrolizumab, Nivolumab, |
| | Atezolizumab, Durvalumab |
| Cutaneous squamous-cell | Cemiplimab, Pembrolizumab, |
| carcinoma | Cosibelimab-ipdl |
| Alveolar soft part sarcoma | Atezolizumab |
| (ASPS) | |
| Basal cell carcinoma | Cemiplimab |
| Biliary tract | Pembrolizumab, Durvalumab |
| Bladder | Atezolizumab, Nivolumab, |
| | Avelumab, Durvalumab, |
| | Pembrolizumab |
| Breast | Atezolizumab, Pembrolizumab |
| dMMR solid cancer | Dostarlimab |
| Endometrial | Dostarlimab, Pembrolizumab |
| Esophagus | Pembrolizumab, Nivolumab |
| Malignant pleural mesothelioma | Pembrolizumab |
| (MPM) | |
| Mesothelioma | Nivolumab |
| Nasopharyngeal | Toripalimab-tpzi |
| Non-Hodgkin's lymphoma | Pembrolizumab |
| Renal Cell Carcinoma | Pembrolizumab, Nivolumab |
| TMB-high cancers | Pembrolizumab |

In another aspect, the cancer is resistant to anti-CTLA-4 therapy and optionally anti-PD-L1 therapy. Exemplary cancers resistant to anti-CTLA-4 therapy and optionally anti-PD-L1 therapy include melanoma, NSCLC, hepatocellular carcinoma, renal cell carcinoma, colorectal cancer, and mesothelioma.

TABLE 2
FDA approved anti-CTLA-4 treatments

| Tumor Type | Target | Drug |
|---|---|---|
| Melanoma | CTLA-4; LAG3/PD-1 | Ipilimumab; |
| | | Nivolumab + |
| | | Relatlimab |
| NSCLC | CTLA-4; CTLA-4/PD-L1 | Ipilimumab; |
| | | Tremelimumab |
| Hepatocellular | CTLA-4; CTLA-4/PD-L1 | Ipilimumab; |
| Carcinoma | | Tremelimumab |
| Renal Cell | CTLA-4 | Ipilimumab |
| Carcinoma | | |
| Colorectal | CTLA-4 | Ipilimumab |
| Mesothelioma | CTLA-4 | Ipilimumab |

Cancers that are known as head and neck cancers usually begin in the squamous cells that line the mucosal surfaces inside the head and neck. They also can begin in the salivary glands. They are also further characterized by the area of the head or neck where they begin, e.g., the oral cavity, the pharynx, the larynx, the paranasal sinuses and nasal cavity and the salivary glands. In some instances, a head and neck cancer described herein is selected from laryngeal and hypopharyngeal cancer, nasal cavity and paranasal sinus cancer, nasopharyngeal cancer, oral and oropharyngeal cancer, or salivary gland cancer.

In some embodiments, a head and neck cancer described herein is a human papillomavirus (HPV)-positive tumor.

In some cases, a head and neck cancer described herein is a metastatic head and neck cancer.

In some cases, a head and neck cancer described herein is a relapsed or refractory head and neck cancer.

In some embodiments, a cancer, e.g., a head and neck cancer, described herein is resistant to a checkpoint inhibitor therapy such as a PD-1 treatment in the absence of the EPHA2 kinase inhibitor. As used herein, resistance means the continued progression of a cancer (further growth/metastasis) while undergoing treatment, either following initial response (acquired) or no initial response (intrinsic).

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In terms of oncology, the treatment can be first line, second line, third line, fourth line or fifth line therapy.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disease or condition, such as one or more adverse symptoms, disorders, illnesses, pathologies, diseases, or complications caused by or associated with the disease or condition, or an improvement in an underlying cause or a consequence of the disease or condition, or a reversal of the disease or condition.

Treatment can therefore result in decreasing, reducing, inhibiting, suppressing, limiting, controlling or preventing a disease or condition, or an associated symptom or consequence, or underlying cause; decreasing, reducing, inhibiting, suppressing, limiting, controlling or preventing a progression or worsening of a disease, condition, symptom or consequence, or underlying cause; or further deterioration or occurrence of one or more additional symptoms of the disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disease or symptom in the subject, such as one or more adverse symptoms, disorders, illnesses, pathologies, diseases, or complications caused by or associated with a disease or condition. Treatment methods affecting one or more underlying causes of the condition, disease or symptom are therefore considered to be beneficial. Stabilizing a disorder or condition is also a successful treatment outcome.

A therapeutic benefit or improvement therefore need not be complete ablation of one, most, or all symptoms, complications, consequences or underlying causes associated with the condition, disorder or disease. Thus, a satisfactory endpoint may be achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the disease or condition, over a short or long duration of time (hours, days, weeks, months, etc.). Responsiveness to therapy includes one or more of: reduction in tumor size, volume or burden, longer time to tumor progression, longer overall survival or reduction in disease progression.

As used herein, the term “effective amount” refers to a dose or concentration of a therapeutic agent that induces a desired pharmacologic and/or physiologic effect. In some instances, the effective amount refers to a dose or concentration of a therapeutic agent that treats or ameliorates a subject's disease or condition. In some cases, the effective amount refers to a dose or concentration of a therapeutic agent that, after administration to a subject, leads to a therapeutic benefit or improvement.

As used herein, the terms “individual(s)”, “subject(s)” and “patient(s)” mean any animal or mammal. In some embodiments, the mammal is a human. In some embodiments, the mammal is a non-human, e.g., canine, feline, bovine, murine, rat, simian, equine, hare, rabbit or leporidae. None of the terms require or are limited to situations characterized by the supervision (e.g. constant or intermittent) of a health care worker (e.g. a veterinarian, a doctor, a registered nurse, a nurse practitioner, a physician's assistant, an orderly or a hospice worker).

The invention is further illustrated by the following non-limiting examples.

### EXAMPLES

### Materials and Methods

Cell Lines: The mouse oral cancer MOC2 cell line was obtained from Dr. Ravindra Uppaluri (Dana-Farber Cancer Institute, Boston, MA) and maintained in DMEM/Ham's F12 media (2:1) with 5% fetal bovine serum (FBS), 1% penicillin/streptomycin, 500 ng/mL hydrocortisone, 0.5 ng/mL murine epidermal growth factor (Thermo Fisher Scientific, Waltham, MA), and 5 ug/mL insulin (MilliporeSigma, St. Louis, MO). The mouse oral cancer FMOC1 cell line was developed in-house and maintained in DMEM with 10% FBS and 1% penicillin/streptomycin.

Immunoprecipitation of EPHA2: Cells were lysed in NP-40 lysis buffer (50 mM HEPES, 150 mM NaCl, 1% NP-40, 0.5% Doxycholic acid, 10% glycerol, 2.5 mM EGTA, 1 mM EDTA, 1 mM DTT, 1 mM PMSF, 1 mM BGP, 1 mM Na₃ VO₄, and 10 μg/mL of leupeptin and aprotinin). Lysates were sheared and centrifuged at 15,000×g for 10 min at 4° C. 250 μg of protein was incubated overnight at 4° C. with 2 ug of primary EPHA2 antibody. Then, 25 μl of protein A/G agarose beads (Santa Cruz Biotechnology) were incubated with the lysate for 3 hr at 4° C. Immunoprecipitates were pelleted by centrifugation and washed 5 times with NP-40 buffer. The captured immunocomplexes were eluted by boiling in 2×SDS sample buffer for 5 min and subjected to immunoblot analysis for p-Tyr and EPHA2.

Immunoblotting: Cells were lysed in RIPA lysis buffer (1M Tris-HCL, pH 7.4, 150 mM NaCl, 500 mM EDTA, 5% DOC, 0.1% Triton™-X100, 1 mM DTT, 1 mM PMSF, 1 mM BGP, 1 mM Na₃ VO₄, and 10 μg/mL of leupeptin and aprotinin). Lysates were subjected to SDS page and analysis for total DDR1, phosphorylated DDR1 (Y792), and GAPDH.

In vivo tumor growth: Animal procedures and maintenance were conducted in accordance with the University of Wisconsin School of Medicine and Public Health IACUC guidelines. Female C57bl/6 mice (4-6 weeks old) were obtained from Charles River Laboratories (Wilmington, MA, USA). Female FVB/NJ mice (4-6 weeks old) were obtained from The Jackson Laboratory (Bar Harbor, ME, USA). 1×10⁶ cells were resuspended in PBS with Matrigel® (50% v/v, R&D systems, #344-005-01) and inoculated by subcutaneous injection bilaterally into the dorsal flank of each mouse. Tumors were measured two times per week using a digital caliper. Tumors were allowed to grow to ~80 mm³ and mice were randomized into groups before initiation of treatment. Either IgG or PD-L1 (10 mg/kg) was administered by intraperitoneal injection every four days. Either PBS or Ensartinib (25 mg/kg in PBS) was administered by oral gavage twice daily.

Tumor dissociation and flow cytometry: Tumors were harvested following euthanasia and placed into a 5 mL tube containing PBS. Each tumor was minced using sterile scissors, mashed through a 70 μM mesh strainer, and rinsed with PBS. Cells were stained with a live/dead dye at 4° C. for 30 minutes before adding flow buffer (PBS without Ca/Mg, 2% FBS, 2 mM EDTA, 25 ug/mL DNase I) containing Fc Block™ (Biolegend, San Diego, CA) and incubating at room temperature for 15 minutes. Cells were then stained with fluorescent antibodies at 4° C. for 30 minutes then washed with flow buffer. Cells were then permeabilized overnight using the FoxP3/Transcription Factor Staining Buffer Kit (Tonbo Biosciences) according to manufacturer instructions. Cells were then stained with fluorescent intracellular antibodies at room temperature for 30 minutes. Cells were washed and resuspended in flow buffer. UltraComp eBeads™ (Thermo Fisher Scientific) were stained with fluorescent antibodies for 30 minutes at 4° C. All samples were run on an Attune™ N×T flow cytometer (Thermo Fisher Scientific). Analysis was performed in FlowJo (BD, Franklin Lakes, NK; RRID: SCR_008520).

Picro Sirius Red staining: Tissue slides were deparaffinized in a series of descending EtOH concentrations. Picro Sirius Red dye (Abcam, was added to tissue sections and incubated at room temperature 1 hour. Slides were then rinsed with two exchanges of acetic acid and two exchanges of 100% EtOH before coverslipping. Slides were imaged on a brightfield microscope at 20× magnification. Mean intensity analysis of Picro Sirius Red stain was performed in FIJI.

Immunofluorescent staining: Tissue slides were deparaffinized in a series of descending EtOH concentrations and antigen retrieval was performed in citrate buffer in a decloaking chamber. Briefly, tissue sections were blocked with normal goat serum and incubated in COL1A1 primary antibody overnight at 4° C. Slides were rinsed and incubated in goat anti-mouse A488 secondary antibody for 90 minutes at room temperature. The tissue was then counterstained with DAPI and coverslipped. Slides were imaged on a Leica confocal microscope at 20× magnification. Mean intensity analysis of COL1A1 signal was performed in FIJI.

Second Harmonic Generation: Tissue slides were deparaffinized in a series of descending EtOH concentrations and antigen retrieval was performed in citrate buffer in a decloaking chamber. Slides were coverslipped and subjected to two-photon imaging on a Bruker multiphoton microscope at 20× magnification. The number of collagen fibers present in each field of view was analyzed using CT-FIRE.

TABLE 3
Flow cytometry antibodies

| | Fluorescent | | Catalog |
|---|---|---|---|
| Antigen | Marker | Vendor | Number |
| CD19 | SuperBright | Thermo Fisher | 67-0193-80 |
| | 702 | Scientific Waltham, | |
| | | MA, USA | |
| CD27 | PE-Vio ®615 | Miltenyi Biotech | 130-123-043 |
| | | Bergisch Gladbach, | |
| | | Germany | |
| CD3 | AlexaFluor ® | Biolegend | 100210 |
| | 488 | San Digeo, CA, USA | |
| CD4 | PerCP-eFluor | Thermo Fisher | 46-0041-82 |
| | 710 | Scientific | |
| | | Waltham, MA, USA | |
| CD69 | PE-Cy5 | Biolegend | 104509 |
| | | San Diego, CA, USA | |
| CD8 | AlexaFluor ® | Biolegend | 100730 |
| | 700 | San Diego, CA, USA | |
| NK1.1 | SuperBright | Thermo Fisher | 62-5941-82 |
| | 436 | Scientific | |
| | | Waltham, MA, USA | |
| Perforin | APC | Biolegend | 154303 |
| | | San Diego, CA, USA | |
| CD11b | PE-Vio ®615 | Miltenyi Biotech | 130-113-807 |
| | | Bergisch Gladbach, | |
| | | Germany | |
| CD11c | SuperBright | Thermo Fisher | 63-0114-82 |
| | 600 | Scientific | |
| | | Waltham, MA, USA | |
| F4/80 | SuperBright | Thermo Fisher | 67-4801-80 |
| | 702 | Scientific | |
| | | Waltham, MA, USA | |
| MHCII (I-Ab) | Brilliant ™ | BD | 744929 |
| | Violet 510 | Franklin Lakes, NJ, | |
| | | USA | |
| CD45 | PE-Cy7 | Biolegend | 103114 |
| | | San Diego, CA, USA | |
| Live/Dead | Ghost Dye ® | Tonbo Biosciences | 13-0865-T100 |
| | Red 780 | San Diego, CA, USA | |

TABLE 4
Flow cytometry gating strategies

| Cell Type | Gating Path^{a} |
|---|---|
| CD8 T cells | FSC Singlets/SSC Singlets/GR780−/CD45+/ |
| | CD3+/CD4−, CD8+ |
| Activated cytotoxic | FSC Singlets/SSC Singlets/GR780−/CD45+/ |
| CD8 T cells | CD3+/CD4−, CD8+/CD69+, Perforin+ |
| CD4 T cells | FSC Singlets/SSC Singlets/GR780−/CD45+/ |
| | CD3+/CD4+, CD8− |
| NKT cells | FSC Singlets/SSC Singlets/GR780−/CD45+/ |
| | CD3+/NK1.1+ |
| NK cells | FSC Singlets/SSC Singlets/GR780−/CD45+/ |
| | CD3−/NK1.1+ |
| Activated NK cells | FSC Singlets/SSC Singlets/GR780−/CD45+/ |
| | CD3−/NK1.1+/CD69+ |
| Cytotoxic NK cells | FSC Singlets/SSC Singlets/GR780−/CD45+/ |
| | CD3−/NK1.1+/CD27+, Perforin+ |
| M2 macrophages | FSC Singlets/SSC Singlets/GR780−/CD45+/ |
| | CD11b+/F480+/CD11c−, MHCII+ |

^{a}Gates separated by a slash (/) indicate a parent/child relationship. Gates separated by a comma (,) indicate a quadrant gate relationship

### Example 1: In Vivo Studies of the Combination of Ensartinib in Combination with Anti-PD-L1 Therapy

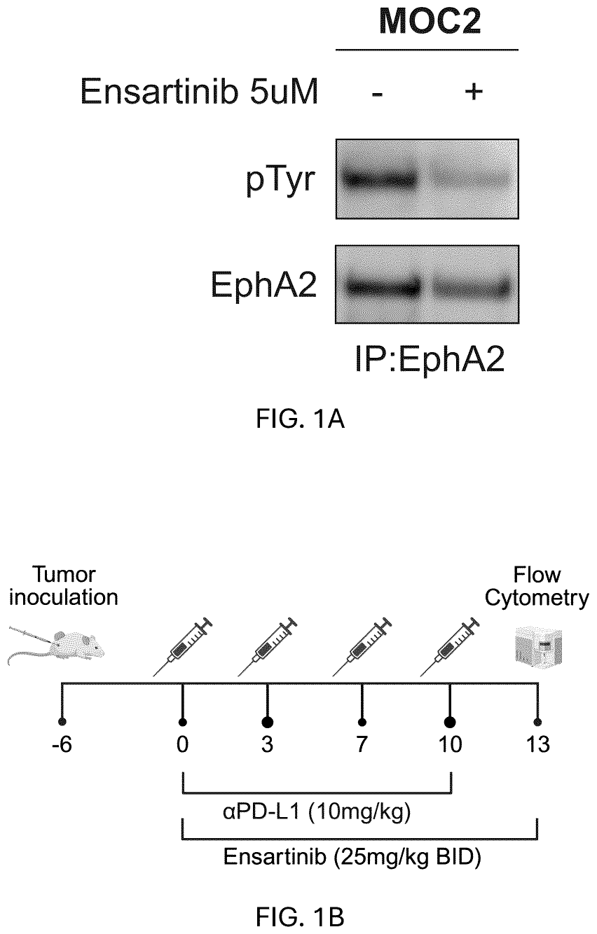

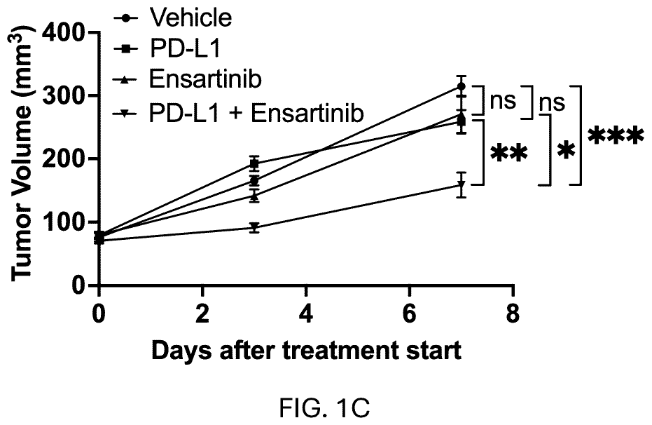

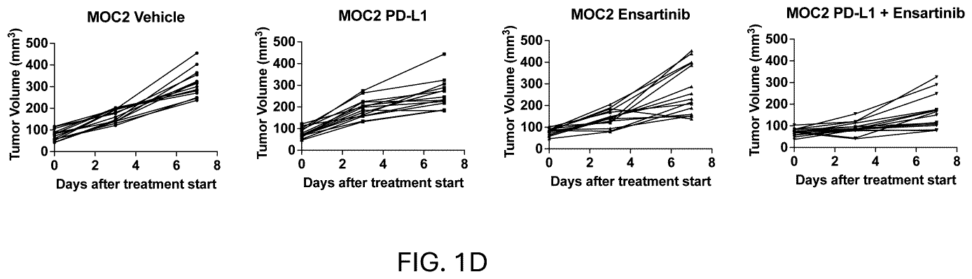

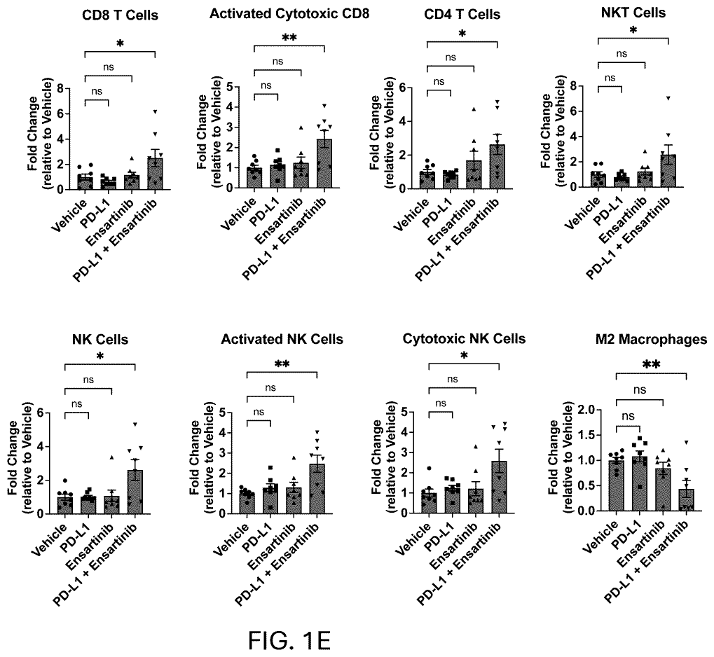

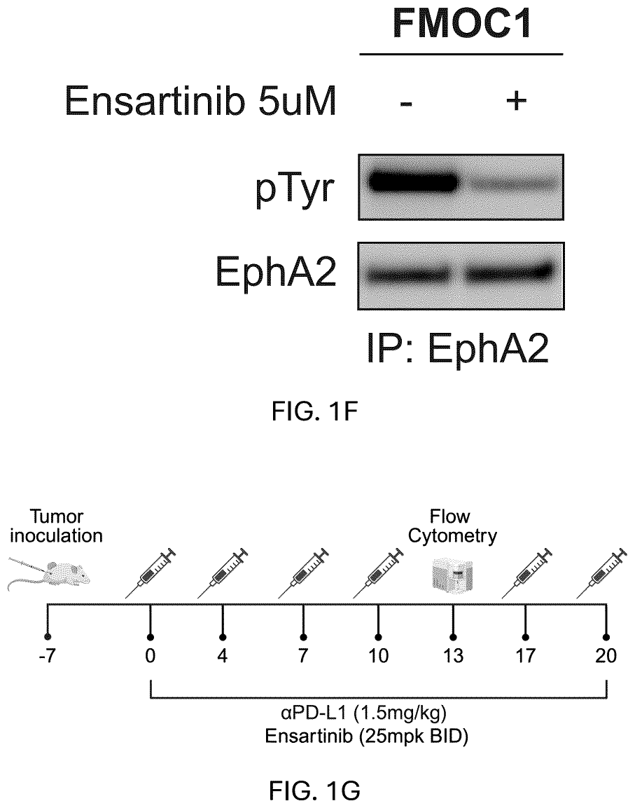

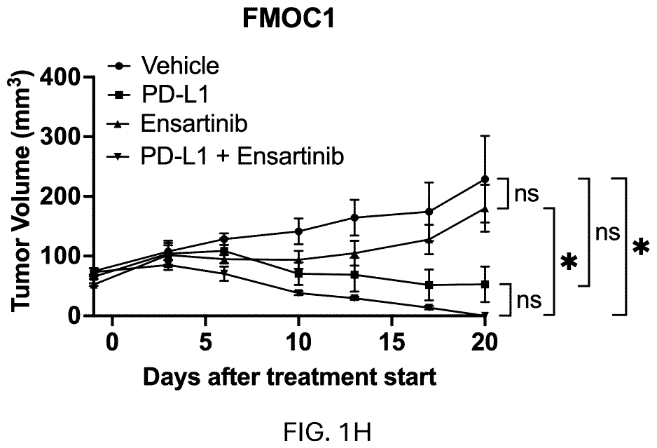

Resistance to anti-PD-1/PD-L1 therapy is a persistent problem in the management of patients with head and neck squamous cell carcinoma (HNSCC). Outlined herein is a role for the receptor tyrosine kinase EPHA2 in mediating this resistance, and that targeting EPHA2 with the small molecule ensartinib in combination with anti-PD-L1 overcomes this resistance. In the MOC2 murine model of HNSCC, which expresses high levels of endogenous EPHA2, the ability of ensartinib to reduce phosphorylation of EPHA2 was demonstrated (FIG. 1A). MOC2 tumor-bearing mice were treated for 13 days with either vehicle control anti-PD-L1 every 3-4 days, ensartinib twice daily, or the combination of anti-PD-L1 and ensartinib (FIG. 1B). In the MOC2 model, no significant anti-tumor effect was observed in groups treated with anti-PD-L1 therapeutic antibody or ensartinib alone compared to vehicle control. A significant delay in tumor growth was observed when the anti-PD-L1 and ensartinib were administered in combination (FIG. 1C, 1D). Further, significant infiltration of anti-tumor CD8, CD4, NKT, NK, and B cells; activation of CD8 and NK cells; and increased cytotoxicity of CD8 and NK cells were observed only in the combination treatment group. Moreover, pro-tumor macrophage populations are reduced in the combination treatment group (FIG. 1E). In the FMOC1 murine model of HNSCC, which expresses high levels of endogenous EPHA2, \ the ability of ensartinib to reduce phosphorylation of EPHA2 was demonstrated (FIG. 1F). FMOC1 tumor-bearing mice were treated for 30 days with either vehicle control, anti-PD-L1 every 3-4 days, ensartinib twice daily, or the combination of anti-PD-L1 and ensartinib (FIG. 1G). The FMOC1 model is moderately sensitive to anti-PD-L1 therapeutic antibody, but insensitive to ensartinib alone compared to vehicle control. Complete regression of all tumors without recurrence was observed when anti-PD-L1 and ensartinib were administered in combination. No tumors in the vehicle group and 50% of tumors in the anti-PD-L1 alone group regressed (FIG. 1H, 1I).

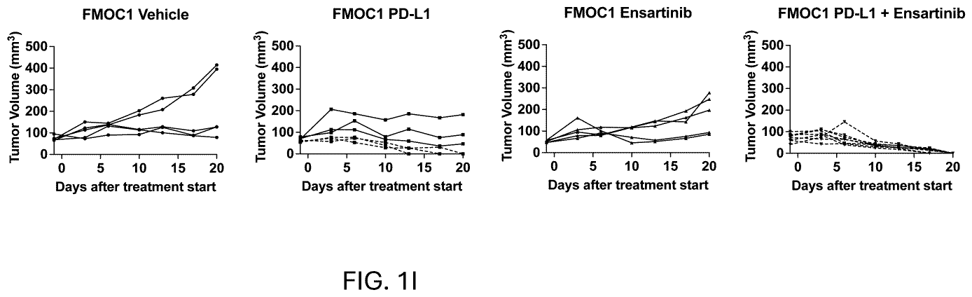

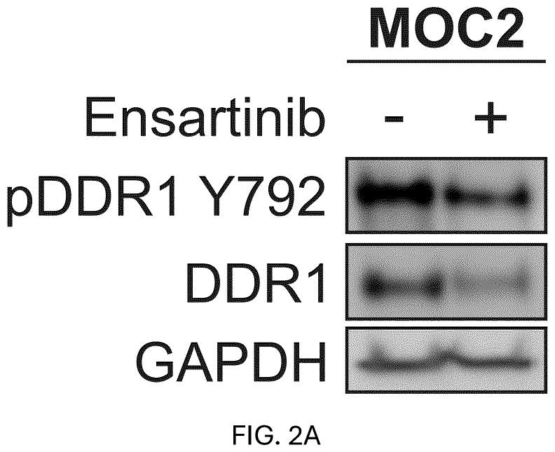

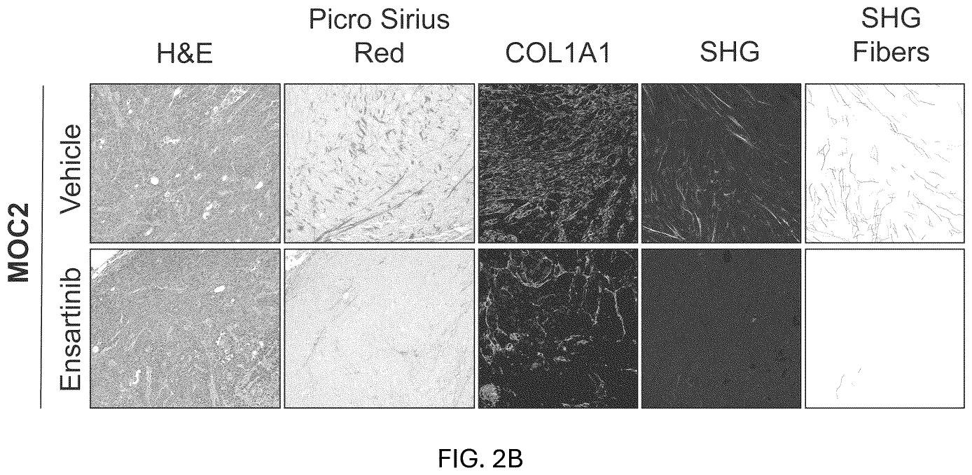

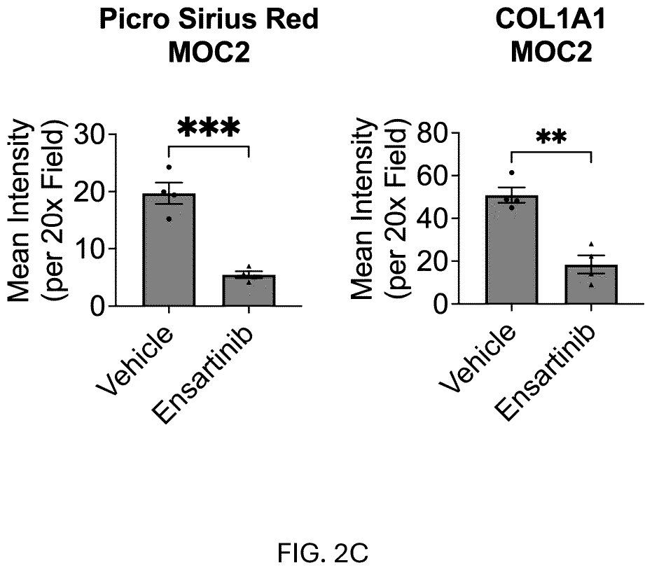

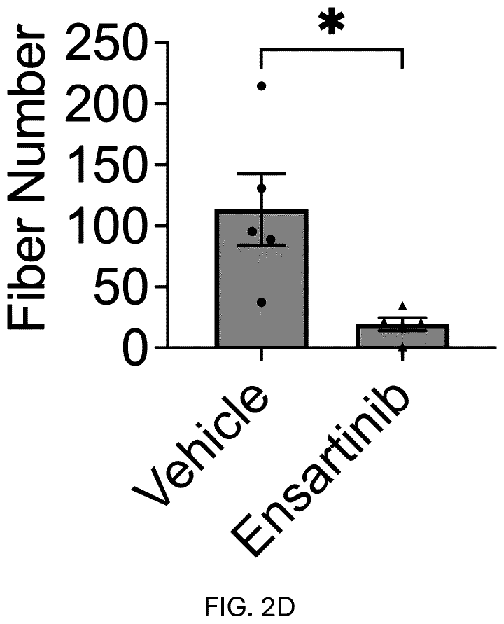

Analysis of collagen in the MOC2 tumor stroma revealed a significant decrease in DDR1 expression (FIG. 2A) and collagen deposition in ensartinib-treated tumors compared to vehicle-treated tumors (FIGS. 2B-D). Together, these data suggest that combination of anti-PD-L1 therapeutic antibody with ensartinib can help overcome resistance to both therapeutic modalities given as monotherapy, and ensartinib may prime tumors to respond to anti-PD-L1 by promoting breakdown of stromal collagen networks via DDR1 to allow influx of anti-tumor immune infiltrate.

### Example 2: Phase I Dose-Escalation Study of Ensartinib in Combination with Pembrolizumab in Patients with Recurrent or Metastatic Head and Neck Cancer

A traditional 3×3 dose-escalation trial of ensartinib and pembrolizumab in patients with recurrent or metastatic disease will be conducted. The objectives are to 1) assess safety and feasibility, 2) evaluate the response rate, 3) progression free survival, and 4) analyze changes in the tumor immune microenvironment (TIME) using Digital Spatial proteomics, single-cell RNA sequencing/CITE-seq, circulating tumor cell (CTC analysis) and multispectral immunofluorescence microscopy in pre- and post-treatment tumor samples. It is expected that all four objectives will be met and the combination of ensartinib and pembrolizumab will improve progression free survival compared to either agent individually.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a cancer that is resistant to anti-PD-1 therapy, anti-PD-L1 therapy, anti-CTLA-4 therapy, or a combination thereof, comprising administering to the patient a combination comprising an EPHA2 receptor tyrosine kinase inhibitor and an immune checkpoint inhibitor,
wherein the immune checkpoint inhibitor comprises an anti-PD-1 inhibitor, an anti-PD-L1 inhibitor, an anti-CTLA-4 inhibitor, or a combination thereof.

2. The method of claim 1, wherein the anti-PD-1 inhibitor or anti-PD-L1 inhibitor is a monoclonal antibody.

3. The method of claim 2, wherein the monoclonal antibody comprises pembrolizumab, cemiplimab, tiselizumab, dostarlimab, retifanlimab, toripalimab, avelumab, durvalumab, cosibelimab or atezolizumab.

4. The method of claim 1, wherein the anti-CTLA-4 inhibitor comprises a monoclonal antibody.

5. The method of claim 4, wherein the anti-CTLA-4 inhibitor comprises ipilimumab, tremelimumab, or a combination thereof.

6. The method of claim 1, wherein the EPHA2 receptor tyrosine kinase inhibitor comprises ensartinib, dasatinib, targefrin, regorafenib, (N-[4-[(4-ethyl-1-piperazinyl)methyl]-3-(trifluoromethyl)phenyl]-4-methyl-3-[[[5-(2-thienyl)-3-pyridinyl]carbonyl]amino]-benzamide), or a combination thereof.

7. The method of claim 1, wherein the EPHA2 receptor tyrosine kinase inhibitor is orally administered.

8. The method of claim 7, wherein the EPHA2 receptor tyrosine kinase inhibitor is administered daily.

9. The method of claim 1, wherein the cancer is head and neck squamous cell carcinoma (HNSCC), the PD-L1 inhibitor is pembrolizumab, and the EPHA2 receptor tyrosine kinase inhibitor is ensartinib.

10. The method of claim 1, wherein the cancer resistant to anti-PD-1 therapy or anti-PD-L1 therapy is a head and neck cancer, melanoma, non-small cell lung cancer, Hodgkins lymphoma, Merkel cell carcinoma, microsatellite instability-high (MSI-H) and mismatch repair deficiency (dMMR), colorectal cancer, gastric cancer, hepatocellular carcinoma, cervical cancer, small cell lung cancer, cutaneous squamous cell carcinoma, alveolar soft part carcinoma, basal cell carcinoma, biliary tract carcinoma, bladder cancer, breast cancer, mismatch repair deficiency (dMMR) solid cancer, endometrial cancer, esophageal cancer, malignant pleural mesothelioma (MPM), mesothelioma, nasopharyngeal cancer, non-Hodgkin's lymphoma, renal cell carcinoma, or a cancer with a high tumor burden (TMB-high cancer).

11. The method of claim 1, wherein the cancer resistant to anti-PD-1 therapy or anti-PDL1-therapy is HNSCC.

12. The method of claim 1, wherein the cancer resistant to anti-CTLA-4 therapy is melanoma, non-small cell lung cancer, hepatocellular carcinoma, renal cell carcinoma, colorectal cancer, or mesothelioma.

13. A method of treating HNSCC that is resistant to anti-PD-1 therapy or anti-PD-L1 therapy, comprising administering a combination comprising ensartinib and pembrolizumab.
